(19) United States
(12) Patent Application Publication
(10) Pub. No.: US 2007/0183982 A1
(43) Pub. Date: Aug. 9, 2007

(54) Pharmaceutical composition for aerosols with two or more active substances and at least one surfactant

(76) Inventors: Erhard Berkel, Ingelheim am Rhein (DE); Hubert Hoelz, Oberheimbach (DE); Friedrich Schmidt, Ingelheim am Rhein (DE)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(21) Appl. No.: 11/668,123

(22) Filed: Jan. 29, 2007

(30) Foreign Application Priority Data
Feb. 9, 2006 (DE) DE 2006 006 207
Nov. 10, 2006 (DE) DE 2006 053 374

(51) Int. Cl. A61K 9/72; A61K 31/56; A61K 31/4745; A61K 31/48

(52) U.S. Cl. 424/40; 514/171; 514/291; 514/337; 514/288; 514/651

(57) ABSTRACT

Disclosed are new pharmaceutical formulations for aerosols with at least two or more active substances together with at least one surfactant for inhalative or nasal application.

This application claims priority benefit to DE 10 2006 006 207, filed Feb. 9, 2006, and DE 10 2006 053 374, filed Nov. 10, 2006, the contents of which are incorporated herein in their entirety.

The present invention relates to new pharmaceutical formulations for aerosols with at least two or more active substances together with at least one surfactant for inhalative or nasal application.

Prior Art

In propellant-driven metered-dose inhalers the active substances may be formulated as a solution or suspension. In the overwhelming majority, aerosol formulations for metered-dose inhalers are provided in the form of a suspension, particularly if the preparation contains more than one active substance. Solution formulations are used only to a limited extent. In these cases, the formulations normally contain only one active substance.

In a suspension, as a rule the chemical stability of the active substances is significantly higher than in solution. Additionally, the active substance may be more concentrated in a suspension than in a solution, which means that higher dosages can be obtained with the suspension formulation.

In suspension formulations it is a major drawback that the suspended particles accumulate over time (e.g. on storage) to form more or less stable, larger aggregates or loose flakes, or they sediment or float or, in the worst case, exhibit particle growth, thereby seriously impairing the pharmaceutical quality of the product. The size of the particles formed or the rate of particle growth are influenced by the solution characteristics of the liquid phase. Thus, the penetration of moisture during storage or an intentional increase in the polarity, e.g. by the addition of cosolvents, may have a disastrous effect on the quality of the medicinal end product, particularly if the suspended particles have polar structural elements. By adding surfactants it is possible to achieve physical stabilisation of the suspension, by reducing the harmful effects of moisture and/or particle growth and enabling suspended particles to be held in suspension for longer.

Solution formulations are naturally unaffected by the problems of increasing particle size of separation processes such as sedimentation or flocculation. However, in this case, chemical breakdown processes present a serious risk. Another disadvantage is that the limited solubility of the ingredients can prevent the administration of high doses. Solvents which have proved particularly suitable in the past include the chlorofluorocarbons TG 11 (trichlorofluoromethane), TG 12 (dichlorodifluoromethane) and TG 114 (dichlorotetrafluoroethane). By adding cosolvents it is possible to increase the solubility of the ingredients. Also, in solution formulations, additional measures usually have to be taken to stabilise the dissolved components chemically.

The propellant gases used hitherto have usually been CFCs, such as e.g. the above-mentioned TG 11. However, as CFCs have been associated with the destruction of the ozone layer, their manufacture and use are being phased out. The desire is to replace them by the use of special fluorinated hydrocarbons (HFA) which are less damaging to the ozone layer but also have completely different solution characteristics. The toxicological profile and physico-chemical properties, such as the vapour pressure, for example, determine which HFAs are suitable for metered-dose aerosols. The most promising examples at present are TG 134a (1,1,2,2-tetrafluoroethane) and TG 227 (1,1,1,2,3,3,3-heptafluoropropane).

For treatment by inhalation, aerosol formulations containing two or more active substance components may be desired. The active substances are formulated in the necessary concentration uniformly as a solution or uniformly as a suspension, which is often connected with problems regarding chemical stability of the achievable concentration of the individual active substances. Major problems arise when one of the active substances cannot be suspended or is unstable in a suspension formulation of this kind or if one of the active substances is chemically unstable or will not dissolve in a solution formulation, particularly when HFA is used as propellant.

One object of the invention is therefore to develop a formulation for metered-dose aerosols with two or more active substances together with at least one surfactant which overcomes the disadvantages mentioned above.

### DESCRIPTION OF THE INVENTION

Surprisingly, it has now been found that two or more active substances can be formulated, together with at least one surfactant, as a solvent and as a suspension side by side in one formulation, and this formulation has improved properties.

The invention relates to a pharmaceutical preparation in the form of stable aerosol formulations with fluorinated hydrocarbons as propellant gas, particularly TG 134a and/or TG 227, which consists of two or more active substances, wherein at least one active substance is formulated as a solution and at least one active substance is formulated as a suspension and moreover the formulation contains at least one surfactant, in order to improve the properties of the formulation. The pharmaceutical preparation according to the invention is used for treatment by inhalation, particularly of diseases of the oral and pharyngeal cavity and the airways, e.g. asthmatic diseases and COPD.

The invention further relates to metered-dose aerosols which contain the pharmaceutical preparation according to the invention.

### DETAILED DESCRIPTION OF THE INVENTION

In one embodiment, a medicinally useful combination of two or more active substances together with at least one surfactant is used for administration by inhalation or by nasal route.

The pharmaceutically active substances, substance formulations or mixtures of substances used may be any inhalable compounds, such as e.g. inhalable macromolecules, as disclosed in EP 1 003 478. Preferably, substances, substance formulations or mixtures of substances which are taken by inhalation are used for treating respiratory complaints.

Particularly preferred in this context are pharmaceutical compositions selected from among the anticholinergics, betamimetics, steroids, phosphodiesterase IV inhibitors, LTD-4-antagonists and EGFR-kinase inhibitors, antiallergics, ergot alkaloid derivatives, triptanes, CGRP antagonists, phosphodiesterase-V inhibitors, and combinations of active substances of this kind, e.g. betamimetics plus anticholinergics or betamimetics plus antiallergics. In the case of combinations at least one of the active substances contains chemically bound water. Anticholinergic-containing active substances are preferably used, as monopreparations or in the form of combined preparations.

The following are specific examples of the active ingredients or the salts thereof:

Anticholinergics to be used are preferably selected from among tiotropium bromide, oxitropium bromide, flutropium bromide, ipratropium bromide, glycopyrronium salts, trospium chloride, tolterodine, tropenol 2,2-diphenylpropionate methobromide, scopine 2,2-diphenylpropionate methobromide, scopine 2-fluoro-2,2-diphenylacetate-methobromide, tropenol 2-fluoro-2,2-diphenylacetate-methobromide, tropenol 3,3′,4,4′-tetrafluorobenzilate methobromide, scopine 3,3′,4,4′-tetrafluorobenzilate methobromide, tropenol 4,4′-difluorobenzilate methobromide, scopine 4,4′-difluorobenzilate methobromide, tropenol 3,3′-difluorobenzilate methobromide, scopine 3,3′-difluorobenzilate methobromide, tropenol 9-hydroxy-fluorene-9-carboxylate methobromide, tropenol 9-fluoro-fluorene-9-carboxylate methobromide, scopine 9-hydroxy-fluorene-9-carboxylate methobromide, scopine 9-fluoro-fluorene-9-carboxylate methobromide, tropenol 9-methyl-fluorene-9-carboxylate methobromide, scopine 9-methyl-fluorene-9-carboxylate methobromide, cyclopropyltropine benzilate methobromide, 2,2-diphenylpropionate cyclopropyltropine methobromide, cyclopropyltropine 9-hydroxy-xanthene-9-carboxylate methobromide, cyclopropyltropine benzilate methobromide, 2,2-diphenylpropionate cyclopropyltropine methobromide, cyclopropyltropine 9-hydroxy-xanthene-9-carboxylate methobromide, cyclopropyltropine 9-methyl-fluorene-9-carboxylate methobromide, cyclopropyltropine 9-methyl-xanthene-9-carboxylate methobromide, cyclopropyltropine 9-hydroxy-fluorene-9-carboxylate methobromide, methyl cyclopropyltropine 4,4′-difluorobenzilate methobromide, tropenol 9-hydroxy-xanthene-9-carboxylate methobromide, scopine 9-hydroxy-xanthene-9-carboxylate methobromide, tropenol 9-methyl-xanthene-9-carboxylate methobromide, scopine 9-methyl-xanthene-9-carboxylate methobromide, tropenol 9-ethyl-xanthene-9-carboxylate methobromide, tropenol 9-difluoromethyl-xanthene-9-carboxylate methobromide and scopine 9-hydroxymethyl-xanthene-9-carboxylate methobromide, optionally in the form of the racemates, enantiomers or diastereomers thereof and optionally in the form of the solvates and/or hydrates thereof.

Betamimetics which may be used are preferably selected from among albuterol, bambuterol, bitolterol, broxaterol, carbuterol, clenbuterol, fenoterol, formoterol, hexoprenaline, ibuterol, indacaterol, isoetharine, isoprenaline, levosalbutamol, mabuterol, meluadrine, metaproterenol, orciprenaline, pirbuterol, procaterol, reproterol, rimiterol, ritodrine, salmeterol, salmefamol, soterenot, sulphonterol, tiaramide, terbutaline, tolubuterol, CHF-1035, HOKU-81, KUL-1248, 3-(4-{6-[2-hydroxy-2-(4-hydroxy-3-hydroxymethyl-phenyl)-ethylamino]-hexyloxy}-butyl)-benzenesulphonamide, 5-[2-(5,6-diethyl-indan-2-ylamino)-1-hydroxy-ethyl]-8-hydroxy-1H-quinolin-2-one, 4-hydroxy-7-[2-{[2-{[3-(2-phenylethoxy)propyl]sulphonyl}ethyl]-amino}ethyl]-2(3H)-benzothiazolone, 1-(2-fluoro-4-hydroxyphenyl)-2-[4-(1-benzimidazolyl)-2-methyl-2-butylamino]ethanol, 1-[3-(4-methoxybenzyl-amino)-4-hydroxyphenyl]-2-[4-(1-benzimidazolyl)-2-methyl-2-butylamino]ethanol, 1-[2H-5-hydroxy-3-oxo-4H-1,4-benzoxazin-8-yl]-2-[3-(4-N,N-dimethylaminophenyl)-2-methyl-2-propylamino]ethanol, 1-[2H-5-hydroxy-3-oxo-4H-1,4-benzoxazin-8-yl]-2-[3-(4-methoxyphenyl)-2-methyl-2-propylamino]ethanol, 1-[2H-5-hydroxy-3-oxo-4H-1,4-benzoxazin-8-yl]-2-[3-(4-n-butyloxyphenyl)-2-methyl-2-propylamino]ethanol, 1-[2H-5-hydroxy-3-oxo-4H-1,4-benzoxazin-8-yl]-2-{4-[3-(4-methoxyphenyl)-1,2,4-triazol-3-yl]-2-methyl-2-butylamino}ethanol, 5-hydroxy-8-(1-hydroxy-2-isopropylaminobutyl)-2H-1,4-benzoxazin-3-(4H)-one, 1-(4-amino-3-chloro-5-trifluoromethylphenyl)-2-tert.-butylamino)ethanol and 1-(4-ethoxycarbonylamino-3-cyano-5-fluorophenyl)-2-(tert.-butylamino)ethanol, optionally in the form of the racemates, enantiomers or diastereomers thereof and optionally in the form of the pharmacologically acceptable acid addition salts, solvates and/or hydrates thereof.

Steroids which may be used are preferably selected from among prednisolone, prednisone, butixocortpropionate, RPR-106541, flunisolide, beclomethasone, triamcinolone, budesonide, fluticasone, mometasone, ciclesonide, rofleponide, ST-126, dexamethasone, (S)-fluoromethyl 6α,9α-difluoro-17α-[(2-furanylcarbonyl)oxy]-11β-hydroxy-16α-methyl-3-oxo-androsta-1,4-diene-17β-carbothionate, (S)-(2-oxo-tetrahydro-furan-3S-yl) 6α,9α-difluoro-11β-hydroxy-16α-methyl-3-oxo-17α-propionyloxy-androsta-1,4-diene-17β-carbothionate and etiprednol-dichloroacetate (BNP-166), optionally in the form of the racemates, enantiomers or diastereomers thereof and optionally in the form of the salts and derivatives thereof, the solvates and/or hydrates thereof.

PDE IV inhibitors which may be used are preferably selected from among enprofyllin, theophyllin, roflumilast, ariflo (cilomilast), CP-325,366, BY343, D-4396 (Sch-351591), AWD-12-281 (GW-842470), N-(3,5-dichloro-1-oxo-pyridin-4-yl)-4-difluoromethoxy-3-cyclopropylmethoxybenzamide, NCS-613, pumafentine, (−)p-[(4aR*,10bS*)-9-ethoxy-1,2,3,4,4a,10b-hexahydro-8-methoxy-2-methylbenzo[s][1,6]naphthyridin-6-yl]-N,N-diisopropylbenzamide, (R)-(+)-1-(4-bromobenzyl)-4-[(3-cyclopentyloxy)-4-methoxyphenyl]-2-pyrrolidone, 3-(cyclopentyloxy-4-methoxyphenyl)-1-(4-N′-[N-2-cyano-5-methyl-isothioureido]benzyl)-2-pyrrolidone, cis[4-cyano-4-(3-cyclopentyloxy-4-methoxyphenyl)cyclohexane-1-carboxylic acid], 2-carbomethoxy-4-cyano-4-(3-cyclopropylmethoxy-4-difluoromethoxyphenyl)cyclohexan-1-one, cis[4-cyano-4-(3-cyclopropylmethoxy-4-difluoromethoxyphenyl)cyclohexan-1-ol], (R)-(+)-ethyl[4-(3-cyclopentyloxy-4-methoxyphenyl)pyrrolidin-2-ylidene]acetate, (S)-(−)-ethyl[4-(3-cyclopentyloxy-4-methoxyphenyl)pyrrolidin-2-ylidene]acetate, CDP840, Bay-198004, D-4418, PD-168787, T-440, T-2585, arofyllin, atizoram, V-11294A, C₁₋₁₀₁₈, CDC-801, CDC-3052, D-22888, YM-58997, Z-15370, 9-cyclopentyl-5,6-dihydro-7-ethyl-3-(2-thienyl)-9H-pyrazolo[3,4-c]-1,2,4-triazolo[4,3-a]pyridine and 9-cyclopentyl-5,6-dihydro-7-ethyl-3-(tert-butyl)-9H-pyrazolo[3,4-c]-1,2,4-triazolo[4,3-a]pyridine, optionally in the form of the racemates, enantiomers or diastereomers thereof and optionally in the form of the pharmacologically acceptable acid addition salts, solvates and/or hydrates thereof.

LTD4-antagonists which may be used are preferably selected from among montelukast, 1-(((R)-(3-(2-(6,7-difluoro-2-quinolinyl)ethenyl)phenyl)-3-(2-(2-hydroxy-2-propyl)phenyl)thio)methylcyclopropane-acetic acid, 1-(((1 (R)-3(3-(2-(2,3-dichlorothieno[3,2-b]pyridin-5-yl)-(E)-ethenyl)phenyl)-3-(2-(1-hydroxy-1-methylethyl)phenyl)propyl)thio)-methyl)cyclopropane-acetic acid, pranlukast, zafirlukast, [2-[[2-(4-tert-butyl-2-thiazolyl)-5-benzofuranyl]oxymethyl]phenyl]acetic acid, MCC-847 (ZD-3523), MN-001, MEN-91507 (LM-1507), VUF-5078, VUF-K-8707 and L-733321, optionally in the form of the racemates, enantiomers or diastereomers thereof, optionally in the form of the pharmacologically acceptable acid addition salts thereof and optionally in the form of the salts and derivatives thereof, the solvates and/or hydrates thereof.

EGFR-kinase inhibitors which may be used are preferably selected from among cetuximab, trastuzumab, ABX-EGF, Mab ICR-62, 4-[(3-chloro-4-fluorophenyl)amino]-6-{[4-(morpholin-4-yl)-1-oxo-2-buten-1-yl]amino}-7-cyclopropylmethoxy-quinazoline, 4-[(R)-(1-phenyl-ethyl)amino]-6-{[4-(morpholin-4-yl)-1-oxo-2-buten-1-yl]amino}-7-cyclopentyloxy-quinazoline, 4-[(3-chloro-4-fluoro-phenyl)amino]-6-{[4-((R)-6-methyl-2-oxo-morpholin-4-yl)-1-oxo-2-buten-1-yl]amino}-7-[(S)-(tetrahydrofuran-3-yl)oxy]-quinazoline, 4-[(3-chloro-4-fluoro-phenyl)amino]-6-[2-((S)-6-methyl-2-oxo-morpholin-4-yl)-ethoxy]-7-methoxy-quinazoline, 4-[(3-chloro-4-fluorophenyl)amino]-6-({4-[N-(2-methoxy-ethyl)-N-methyl-amino}-1-oxo-2-buten-1-ylamino)-7-cyclopropylmo-methoxy-quinazoline, 4-[(R)-(1-phenyl-ethyl)amino]-6-({4-[N-(tetrahydropyran-4-yl)-N-methyl-amino]-1-oxo-2-buten-1-yl}amino)-7-cyclopropylmethoxy-quinazoline, 4-[(3-chloro-4-fluorophenyl)amino]-6-({4-[N-(2-methoxy-ethyl)-N-methyl-amino]-1-oxo-2-buten-1-yl}amino)-7-cyclopentyloxy-quinazoline, 4-[(3-chloro-4-fluorophenyl)amino]-6-{[4-(N,N-dimethylamino)-1-oxo-2-buten-1-yl]amino}-7-[(R)-(tetrahydrofuran-2-yl)methoxy]-quinazoline, 4-[(3-ethynyl-phenyl)amino]-6,7-bis-(2-methoxy-ethoxy)-quinazoline, 4-[(R)-(1-phenyl-ethyl)amino]-6-(4-hydroxy-phenyl)-7H-pyrrolo[2,3-d]pyrimidine, 3-cyano-4-[(3-chloro-4-fluorophenyl)amino]-6-{[4-(N,N-dimethylamino)-1-oxo-2-buten-1-yl]amino}-7-ethoxy-quinoline, 4-[(R)-(1-phenyl-ethyl)amino]-6-{[4-((R)-6-methyl-2-oxo-morpholin-4-yl)-1-oxo-2-buten-1-yl]amino}-7-methoxy-quinazoline, 4-[(3-chloro-4-fluorophenyl)amino]-6-{[4-(morpholin-4-yl)-1-oxo-2-buten-1-yl]amino}-7-[(tetrahydrofuran-2-yl)methoxy]-quinazoline, 4-[(3-ethynyl-phenyl)amino]-6-{[4-(5,5-dimethyl-2-oxo-morpholin-4-yl)-1-oxo-2-buten-1-yl]amino}-quinazoline, 4-[(3-chloro-4-fluoro-phenyl)amino]-6-{2-[4-(2-oxo-morpholin-4-yl)-piperidin-1-yl]-ethoxy}-7-methoxy-quinazoline, 4-[(3-chloro-4-fluoro-phenyl)amino]-6-(trans-4-amino-cyclohexan-1-yloxy)-7-methoxy-quinazoline, 4-[(3-chloro-4-fluoro-phenyl)amino]-6-(trans-4-methanesulphonylamino-cyclohexan-1-yloxy)-7-methoxy-quinazoline, 4-[(3-chloro-4-fluoro-phenyl)amino]-6-(tetrahydropyran-3-yloxy)-7-methoxy-quinazoline, 4-[(3-chloro-4-fluoro-phenyl)amino]-6-{1-[(morpholin-4-yl)carbonyl]-piperidin-4-yloxy}-7-methoxy-quinazoline, 4-[(3-chloro-4-fluoro-phenyl)amino]-6-(piperidin-3-yloxy)-7-methoxy-quinazoline, 4-[(3-chloro-4-fluoro-phenyl)amino]-6-[1-(2-acetylamino-ethyl)-piperidin-4-yloxy]-7-methoxy-quinazoline, 4-[(3-chloro-4-fluoro-phenyl)amino]-6-(tetrahydropyran-4-yloxy)-7-ethoxy-quinazoline, 4-[(3-chloro-4-fluoro-phenyl)amino]-6-{trans-4-[(morpholin-4-yl)carbonylamino]-cyclohexan-1-yloxy}-7-methoxy-quinazoline, 4-[(3-chloro-4-fluoro-phenyl)amino]-6-{1-[(piperidin-1-yl)carbonyl]-piperidin-4-yloxy}-7-methoxy-quinazoline, 4-[(3-chloro-4-fluoro-phenyl)amino]-6-(cis-4-{N-[(morpholin-4-yl)carbonyl]-N-methyl-amino}-cyclohexan-1-yloxy)-7-methoxy-quinazoline, 4-[(3-chloro-4-fluoro-phenyl)amino]-6-(trans-4-ethanesulphonylamino-cyclohexan-1-yloxy)-7-methoxy-quinazoline, 4-[(3-chloro-4-fluoro-phenyl)amino]-6-(1-methanesulphonyl-piperidin-4-yloxy)-7-(2-methoxy-ethoxy)-quinazoline, 4-[(3-chloro-4-fluoro-phenyl)amino]-6-[1-(2-methoxy-acetyl)-piperidin-4-yloxy]-7-(2-methoxy-ethoxy)-quinazoline, 4-[(3-ethynyl-phenyl)amino]-6-(tetrahydropyran-4-yloxy]-7-methoxy-quinazoline, 4-[(3-chloro-4-fluoro-phenyl)amino]-6-(cis-4-{N-[(piperidin-1-yl)carbonyl]-N-methyl-amino}-cyclohexan-1-yloxy)-7-methoxy-quinazoline, 4-[(3-chloro-4-fluoro-phenyl)amino]-6-{cis-4-[(morpholin-4-yl)carbonylamino]-cyclohexan-1-yloxy}-7-methoxy-quinazoline, 4-[(3-chloro-4-fluoro-phenyl)amino]-6-{1-[2-(2-oxopyrrolidin-1-yl)ethyl]-piperidin-4-yloxy}-7-methoxy-quinazoline, 4-[(3-ethynyl-phenyl)amino]-6-(1-acetyl-piperidin-4-yloxy)-7-methoxy-quinazoline, 4-[(3-ethynyl-phenyl)amino]-6-(1-methyl-piperidin-4-yloxy)-7-methoxy-quinazoline, 4-[(3-ethynyl-phenyl)amino]-6-(1-methanesulphonyl-piperidin-4-yloxy)-7-methoxy-quinazoline, 4-[(3-chloro-4-fluoro-phenyl)amino]-6-(1-methyl-piperidin-4-yloxy)-7-(2-methoxy-ethoxy)-quinazoline, 4-[(3-ethynyl-phenyl)amino]-6-{1-[(morpholin-4-yl)carbonyl]-piperidin-4-yloxy}-7-methoxy-quinazoline, 4-[(3-chloro-4-fluoro-phenyl)amino]-6-{1-[(N-methyl-N-2-methoxyethyl-amino)carbonyl]-piperidin-4-yloxy}-7-methoxy-quinazoline, 4-[(3-chloro-4-fluoro-phenyl)amino]-6-(1-ethyl-piperidin-4-yloxy)-7-methoxy-quinazoline, 4-[(3-chloro-4-fluoro-phenyl)amino]-6-[cis-4-(N-methanesulphonyl-N-methyl-amino)-cyclohexan-1-yloxy]-7-methoxy-quinazoline, 4-[(3-chloro-4-fluoro-phenyl)amino]-6-[cis-4-(N-acetyl-N-methyl-amino)-cyclohexan-1-yloxy]-7-methoxy-quinazoline, 4-[(3-chloro-4-fluoro-phenyl)amino]-6-(trans-4-methylamino-cyclohexan-1-yloxy)-7-methoxy-quinazoline, 4-[(3-chloro-4-fluoro-phenyl)amino]-6-[trans-4-(N-methanesulphonyl-N-methyl-amino)-cyclohexan-1-yloxy]-7-methoxy-quinazoline, 4-[(3-chloro-4-fluoro-phenyl)amino]-6-(trans-4-dimethylamino-cyclohexan-1-yloxy)-7-methoxy-quinazoline, 4-[(3-chloro-4-fluoro-phenyl)amino]-6-(trans-4-{N-[(morpholin-4-yl)carbonyl]-N-methyl-amino}-cyclohexan-1-yloxy)-7-methoxy-quinazoline, 4-[(3-chloro-4-fluoro-phenyl)amino]-6-[2-(2,2-dimethyl-6-oxo-morpholin-4-yl)-ethoxy]-7-[(S)-(tetrahydrofuran-2-yl)methoxy]-quinazoline, 4-[(3-chloro-4-fluoro-phenyl)amino]-6-(1-methanesulphonyl-piperidin-4-yloxy)-7-methoxy-quinazoline, 4-[(3-chloro-4-fluoro-phenyl)amino]-6-(1-cyano-piperidin-4-yloxy)-7-methoxy-quinazoline, and 4-[(3-chloro-4-fluoro-phenyl)amino]-6-{1-[(2-methoxyethyl)carbonyl]-piperidin-4-yloxy}-7-methoxy-quinazoline, optionally in the form of the racemates, enantiomers or diastereomers thereof, optionally in the form of the pharmacologically acceptable acid addition salts thereof, the solvates and/or hydrates thereof.

By acid addition salts with pharmacologically acceptable acids which the compounds may be capable of forming are meant, for example, salts selected from among the hydrochloride, hydrobromide, hydriodide, hydrosulphate, hydrophosphate, hydromethanesulphonate, hydronitrate, hydromaleate, hydroacetate, hydrobenzoate, hydrocitrate, hydrofumarate, hydrotartrate, hydroxalate, hydrosuccinate, hydrobenzoate and hydro-p-toluenesulphonate, preferably the hydrochloride, hydrobromide, hydrosulphate, hydrophosphate, hydrofumarate and hydromethanesulphonate.

Examples of antiallergics are: disodium cromoglycate, nedocromil.

Examples of ergot alkaloids are: dihydroergotamine, ergotamine.

Examples of substances suitable for inhalation include medicaments, medicament formulations and mixtures containing the above-mentioned active substances, and the salts and esters thereof and combinations of these active substances, salts and esters.

Which of the above-mentioned active substances are formulated as a solution and which as a suspension in the preparation according to the invention depends on the particular combinations of active substances and can be determined relatively quickly by solution and suspension experiments.

In a preferred embodiment one or more of the following active substances are suspended: budesonide, cromoglycic acid, nedocromil, reproterol and/or salbutamol (albuterol) or esters, salts and/or solvates derived from these compounds and one or more of the following substances are dissolved: beclomethasone, fenoterol, ipratropium bromide, orciprenaline and/or oxitropium bromide, N-[[2,2-dimethyl-4-(2-oxo-2H-pyridin-1-yl)-6-trifluoromethyl-2H-1-benzopyran-3-yl]methyl]-N-hydroxy-acetamide or esters, salts and/or solvates derived from these compounds. Embodiments containing two different active substances are preferred.

Preferably, the pharmaceutical preparation contains a combination of active substances selected from among the following: beclomethasone, budesonide, cromoglycic acid, fenoterol, flunisolide, fluticasone, ipratropium, nedocromil orciprenaline, oxitropium bromide, reproterol, salbutamol, salmeterol (albuterol), terbutaline, N-[[2,2-dimethyl-4-(2-oxo-2H-pyridin-1-yl)-6-trifluoromethyl-2H-1-benzopyran-3-yl]methyl]-N-hydroxy-acetamide, the esters, salts and/or solvates thereof

A particularly preferred embodiment of the pharmaceutical preparation contains dissolved ipratropium bromide monohydrate, particularly in combination with salbutamol sulphate (albuterol sulphate) as a suspended active substance.

In all the embodiments, the active substances are used in a therapeutically effective amount, i.e. in an amount which can provide successful treatment. The concentration of the active substances and the volume delivered per spray jet are adjusted so that one or just a few spray jets deliver the medicinally necessary or recommended amount of the active substance in question.

One embodiment relates to formulations in which the suspended particles are stabilised by the addition of surfactants. This has the advantage that the particle size remains pharmaceutically stable and acceptable even over lengthy periods, e.g. during storage. Particle sizes of up to 20 μm are preferred, while particle sizes between 5 and 15 μm are most particularly preferred, and most favourably do not exceed 10 μm. The advantage of these particles sizes are that the particles are small enough to penetrate deeply into the lungs, but not so small as to be breathed out again with the exchanged air.

Suitable surfactants include all pharmacologically acceptable substances that have a lipophilic hydrocarbon group and one or more functional hydrophilic group(s). Particularly suitable are C₅₋₂₀-fatty alcohols, C₅₋₂₀-fatty acids, C₅₋₂₀-fatty acid esters, lecithin, glycerides, propyleneglycolesters, polyoxyethylenes, polysorbates, sorbitan esters and/or carbohydrates. C₅₋₂₀-Fatty acids, propyleneglycoldiesters and/or triglycerides and/or sorbitans of C₅₋₂₀-fatty acids are preferred, while sodium or potassium salts of a C₅₋₂₀-fatty acid, an oleic acid and sorbitan mono-, di- or trioleates, a polyvinylpyrrolidone, a polyvinylalcohol, a polyoxyethylenesorbitanester, a polyoxyethyleneglycerol ester, a polyoxyethylene fatty acid ester, a polyoxypropylene fatty acid ester, a polyoxyethylene/polyoxypropylene block copolymer, an alkylpolyglycoside, a benzalkonium chloride and/or a cetylpyridinium chloride are particularly preferred.

Most particularly preferred are polyvinylpyrrolidone K25 (Povidone 25®), polyoxyethylene-20-sorbitan monolaurate, polyoxyethyleneglycerol trioleate or a combination of these surfactants. Particularly preferred according to the invention are polyoxyethylene-20-sorbitan monolaurate and polyoxyethyleneglycerol trioleate, which are on the market and obtainable under the brand names Tween® 20 and Tagat® TO V.

The surfactants are preferably present in the formulations according to the invention in a concentration of 0.001 to 5% (m/m), particularly preferably from 0.01 to 3% (m/m).

In a particularly preferred embodiment of the invention one or more, preferably one of the above-mentioned surfactants are present in a concentration of 0.02 to 0.2% (m/m), preferably from 0.05 to 0.15% (m/m), particularly 0.1% (m/m).

In another preferred alternative embodiment of the invention, one or more, preferably one of the above-mentioned surfactants is present in a concentration of 0.3 to 2.5% (m/m), preferably 0.4 to 2% (m/m), particularly preferably 0.5 to 1.5% (m/m), more preferably 0.75 to 1.25% (m/m), particularly 1.0% (m/m).

A further advantage of the said surfactants is that they can also be used as valve lubricants. Thereof, one embodiment relates to formulations in which said surfactants are added as valve lubricants.

In another embodiment, the solubility of the active substance(s) to be dissolved is increased by the addition of cosolvents. This has the advantage that the active substance(s) to be dissolved can be formulated in a higher concentration. The addition of cosolvents must not cause the liquid phase to exceed the critical polarity threshold above which one of the disadvantages described above occurs to the suspended particles of active substance.

Suitable cosolvents are pharmacologically acceptable alcohols such as ethanol, esters or water or mixtures thereof, ethanol is preferred. The concentration of the cosolvent based on the formulation as a whole may be 0.0001 to 50% (m/m), preferably 0.01 to 25% (m/m). In a preferred embodiment the concentration of cosolvent is 1 to 20% (m/m), preferably 5 to 15% (m/m). Most particularly preferred are those formulations according to the invention in which the concentration of cosolvent is 8 to 12% (m/m), particularly 10% (m/m).

The concentrations specified within the scope of the present invention are always percent by mass [% m/m] based on the mass of the formulation as a whole.

In another embodiment other common propellant gases are added to the HFA propellant gas. Such added propellant gases may be, apart from other fluorinated hydrocarbons, saturated lower hydrocarbons such as propane, butane, isobutane or pentane, provided that the mixture is pharmacologically safe.

In one embodiment, stabilisers are added to the formulation, advantageously affecting the pharmaceutical stability of the active substances even over long periods, e.g. during storage. In the context of the invention, the term stabilisers denotes substances that extend the durability and useful life of the pharmaceutical preparation by preventing or delaying chemical changes to the individual ingredients, particularly the active substances, but also the other additives, e.g. as a result of secondary reactions or degradation, or prevent biological contamination. In this sense, preferred stabilisers are those which affect the pH value of the liquid phase, such as e.g. acids and/or the salts thereof. Particularly suitable acids are hydrochloric acid, sulphuric acid, nitric acid, phosphoric acid, ascorbic acid, citric acid and the salts thereof. Examples of suitable bactericides, fungicides, etc. include benzalkonium chloride and ethylenediamine tetraacetate. Citric acid is most preferred. The concentration of the above-mentioned stabilisers is preferably in the range from 0.0001 to 0.02% (m/m), preferably in the range from 0.0005 to 0.01% (m/m). Particularly preferred formulations according to the invention contain the above-mentioned stabilisers in a concentration of 0.001 to 0.008% (m/m), while a content of 0.002 to 0.006% (m/m), particularly about 0.004% (m/m) is particularly important according to the invention.

A particularly preferred embodiment comprises suspended salbutamol sulphate (albuterol sulphate), dissolved ipratropium bromide, ethanol as cosolvent and citric acid as stabiliser. These particularly preferred formulations according to the invention preferably contain the active substance salbutamol sulphate in a concentration of 0.1 to 0.3% (m/m), particularly preferably 0.15 to 0.25% (m/m), more preferably 0.18 to 0.22% (m/m). These particularly preferred formulations according to the invention also contain ipratropium bromide monohydrate in a concentration of preferably 0.02 to 0.05% (m/m), particularly preferably 0.03 to 0.04% (m/m). Particularly preferred are those compositions according to the invention wherein the ratio of the above-mentioned concentrations of the two active substances salbutamol sulphate und ipratropium bromide monohydrate is in the range from 5:1 to 6:1, particularly preferably in the range from 5.5:1 to 5.9:1. Compositions according to the invention wherein the ratio of the concentrations of the two active substances salbutamol sulphate and ipratropium bromide monohydrate is in the range from 5.60:1 to 5.85:1, particularly in a range from 5.70:1 to 5.80:1 are particularly preferred.

In all the embodiments the formulations are transferred into suitable metal containers for metered-dose aerosols: the metal containers are sealed with suitable metering valves.

Examples of suitable metal containers include the stainless steel one-piece cans (DIN 1.4539) made by Presspart Manufacturing Ltd., Blackburn UK, with a nominal volume of 17 ml. Suitable metering valves include for example BK 357 or BK 361 made by Bespak Europe Ltd., King's Lynn, UK.

The metered-dose aerosol according to the invention preferably contains a pharmaceutical preparation containing a combination of active substances selected from the following group: beclomethasone, budesonide, cromoglycic acid, fenoterol, flunisolide, fluticasone, ipratropium, nedocromil orciprenaline, oxitropium bromide, reproterol, salbutamol, salmeterol (albuterol), terbutaline, N-[[2,2-dimethyl-4-(2-oxo-2H-pyridin-1-yl)-6-trifluoromethyl-2H-1-benzopyran-3-yl]methyl]-N-hydroxy-acetamide, the esters, salts and/or solvates thereof.

Most particularly preferably, the metered-dose aerosol according to the invention contains a pharmaceutical preparation which contains a combination of the active substances salbutamol sulphate (albuterol sulphate) and ipratropium bromide monohydrate.

### EXAMPLES

### Example 1

| | Mass per container | Concentration |
|---|---|---|
| Component | [g] | [% m/m] |
| salbutamol sulphate | 0.0312 | 0.210 |
| ipratropium bromide monohydrate | 0.0055 | 0.037 |
| absolute ethanol | 1.4824 | 10.000 |
| polyoxyethylene-20-sorbitan | 0.0741 | 0.500 |
| monolaurate | | |
| anhydrous citric acid | 0.0006 | 0.004 |
| 1,1,1,2-tetrafluoroethane | 13.2302 | 89.249 |
| (HFA 134a) | | |
| total | 14.8240 | 100.000 |

### Example 2

| | Mass per container | Concentration |
|---|---|---|
| Component | [g] | [% m/m] |
| salbutamol sulphate | 0.0312 | 0.211 |
| ipratropium bromide monohydrate | 0.0055 | 0.037 |
| absolute ethanol | 1.4818 | 10.000 |
| polyoxyethylene-20-sorbitan | 0.1482 | 1.000 |
| monolaurate | | |
| anhydrous citric acid | 0.0006 | 0.004 |
| 1,1,1,2-tetrafluoroethane | 13.1508 | 88.749 |
| (HFA 134a) | | |
| total | 14.81800 | 100.000 |

### Example 3

| | Mass per container | Concentration |
|---|---|---|
| Component | [g] | [% m/m] |
| salbutamol sulphate | 0.0312 | 0.211 |
| ipratropium bromide monohydrate | 0.0055 | 0.037 |
| absolute ethanol | 1.4805 | 10.000 |
| polyoxyethylene-20-sorbitan | 0.2961 | 2.000 |
| monolaurate | | |
| anhydrous citric acid | 0.0006 | 0.004 |
| 1,1,1,2-tetrafluoroethane | 12.9912 | 87.748 |
| (HFA 134a) | | |
| total | 14.8050 | 100.000 |

### Example 4

| | Mass per container | Concentration |
|---|---|---|
| Component | [g] | [% m/m] |
| salbutamol sulphate | 0.0312 | 0.185 |
| ipratropium bromide monohydrate | 0.0055 | 0.032 |
| absolute ethanol | 1.6874 | 10.000 |
| polyoxyethylene-20-sorbitan | 0.0844 | 0.500 |
| monolaurate | | |
| anhydrous citric acid | 0.0007 | 0.004 |
| 1,1,1,2,3,3,3-heptafluoropropane | 15.0649 | 89.279 |
| (HFA 227) | | |
| total | 16.87400 | 100.000 |

### Example 5

| | Mass per container | Concentration |
|---|---|---|
| Component | [g] | [% m/m] |
| salbutamol sulphate | 0.0312 | 0.185 |
| ipratropium bromide monohydrate | 0.0055 | 0.032 |
| absolute ethanol | 1.6853 | 10.000 |
| polyoxyethylene-20-sorbitan | 0.1685 | 1.000 |
| monolaurate | | |
| anhydrous citric acid | 0.0007 | 0.004 |
| 1,1,1,2,3,3,3-heptafluoropropane | 14.9618 | 88.778 |
| (HFA 227) | | |
| total | 16.85300 | 100.000 |

### Example 6

| | Mass per container | Concentration |
|---|---|---|
| Component | [g] | [% m/m] |
| salbutamol sulphate | 0.0312 | 0.186 |
| ipratropium bromide monohydrate | 0.0055 | 0.032 |
| absolute ethanol | 1.6810 | 10.000 |
| polyoxyethylene-20-sorbitan | 0.3362 | 2.000 |
| monolaurate | | |
| anhydrous citric acid | 0.0007 | 0.004 |
| 1,1,1,2,3,3,3-heptafluoropropane | 14.7555 | 87.778 |
| (HFA 227) | | |
| total | 16.81000 | 100.000 |

### Example 7

| | Mass per container | Concentration |
|---|---|---|
| Component | [g] | [% m/m] |
| salbutamol sulphate | 0.0312 | 0.210 |
| ipratropium bromide monohydrate | 0.0055 | 0.037 |
| absolute ethanol | 1.4824 | 10.000 |
| polyoxyethyleneglycerol trioleate | 0.0741 | 0.500 |
| anhydrous citric acid | 0.0006 | 0.004 |
| 1,1,1,2-tetrafluoroethane | 13.2302 | 89.249 |
| (HFA 134a) | | |
| total | 14.8240 | 100.000 |

### Example 8

| | Mass per container | Concentration |
|---|---|---|
| Component | [g] | [% m/m] |
| salbutamol sulphate | 0.0312 | 0.211 |
| ipratropium bromide monohydrate | 0.0055 | 0.037 |
| absolute ethanol | 1.4818 | 10.000 |
| polyoxyethyleneglycerol trioleate | 0.1482 | 1.000 |
| anhydrous citric acid | 0.0006 | 0.004 |
| 1,1,1,2-tetrafluoroethane | 13.1508 | 88.749 |
| (HFA 134a) | | |
| total | 14.81800 | 100.000 |

### Example 9

| | Mass per container | Concentration |
|---|---|---|
| Component | [g] | [% m/m] |
| salbutamol sulphate | 0.0312 | 0.211 |
| ipratropium bromide monohydrate | 0.0055 | 0.037 |
| absolute ethanol | 1.4805 | 10.000 |
| polyoxyethyleneglycerol trioleate | 0.2961 | 2.000 |
| anhydrous citric acid | 0.0006 | 0.004 |
| 1,1,1,2-tetrafluoroethane | 12.9912 | 87.748 |
| (HFA 134a) | | |
| total | 14.8050 | 100.000 |

### Example 10

| | Mass per container | Concentration |
|---|---|---|
| Component | [g] | [% m/m] |
| salbutamol sulphate | 0.0312 | 0.185 |
| ipratropium bromide monohydrate | 0.0055 | 0.032 |
| absolute ethanol | 1.6874 | 10.000 |
| polyoxyethyleneglycerol trioleate | 0.0844 | 0.500 |
| anhydrous citric acid | 0.0007 | 0.004 |
| 1,1,1,2,3,3,3-heptafluoropropane | 15.0649 | 89.279 |
| (HFA 227) | | |
| total | 16.87400 | 100.000 |

### Example 11

| | Mass per container | Concentration |
|---|---|---|
| Component | [g] | [% m/m] |
| salbutamol sulphate | 0.0312 | 0.185 |
| ipratropium bromide monohydrate | 0.0055 | 0.032 |
| absolute ethanol | 1.6853 | 10.000 |
| polyoxyethyleneglycerol trioleate | 0.1685 | 1.000 |
| anhydrous citric acid | 0.0007 | 0.004 |
| 1,1,1,2,3,3,3-heptafluoropropane | 14.9618 | 88.778 |
| (HFA 227) | | |
| total | 16.85300 | 100.000 |

### Example 12

| | Mass per container | Concentration |
|---|---|---|
| Component | [g] | [% m/m] |
| salbutamol sulphate | 0.0312 | 0.186 |
| ipratropium bromide monohydrate | 0.0055 | 0.032 |
| absolute ethanol | 1.6810 | 10.000 |
| polyoxyethyleneglycerol trioleate | 0.3362 | 2.000 |
| anhydrous citric acid | 0.0007 | 0.004 |
| 1,1,1,2,3,3,3-heptafluoropropane | 14.7555 | 87.778 |
| (HFA 227) | | |
| total | 16.81000 | 100.000 |

1. A pharmaceutical preparation for propellant-driven metered-dose aerosols having a fluorinated hydrocarbon (HFA) as propellant, which contains a combination of two or more active substances, wherein at least one active substance is present in dissolved form as well as at least one other active substance in the form of suspended particles together with at least one surfactant.

2. The pharmaceutical preparation according to claim 1, wherein the active substance combination consists of two active substances.

3. The pharmaceutical preparation according to claim 2, wherein the propellant is TG 134a and/or TG 227.

4. The pharmaceutical preparation according to claim 3, wherein the preparation contains a cosolvent.

5. The pharmaceutical preparation according to claim 4, wherein the cosolvent contains one or more pharmacologically acceptable alcohols, a pharmacologically acceptable ester, water or mixtures thereof.

6. The pharmaceutical preparation according to claim 5, wherein the cosolvent is ethanol.

7. The pharmaceutical preparation according to claim 6, wherein the cosolvent is present in a concentration of 0.0001 to 50% (m/m) based on the formulation as a whole.

8. The pharmaceutical preparation according to claim 7, wherein the cosolvent is present in a concentration of 5 to 15% (m/m) based on the formulation as a whole.

9. The pharmaceutical preparation according to claim 8, wherein the preparation is stabilised by a stabiliser.

10. The pharmaceutical preparation according to claim 9, wherein the stabiliser contains one or more acid(s) and/or salt(s) thereof.

11. The pharmaceutical preparation according to claim 9, wherein the stabiliser or stabilisers contain(s) hydrochloric acid, sulphuric acid, nitric acid, phosphoric acid, ascorbic acid, citric acid, benzalkonium chloride and/or ethylenediamine tetraacetate and/or a salt thereof.

12. The pharmaceutical preparation according to claim 9, wherein the stabiliser is citric acid.

13. The pharmaceutical preparation according to claim 11, wherein the stabiliser is present in a concentration of 0.0001 to 0.02% (m/m), based on the formulation as a whole.

14. The pharmaceutical preparation according to claim 13, wherein the stabiliser is present in a concentration of 0.0001 to 0.008% (m/m), based on the formulation as a whole.

15. The pharmaceutical preparation according to claim 14, wherein the preparation contains at least one surfactant.

16. The pharmaceutical preparation according to claim 15, wherein the surfactant is a sodium or potassium salt of a C₅₋₂₀-fatty acid, an oleic acid, a polyvinylpyrrolidone, a polyvinylalcohol, a polyoxyethylenesorbitanester, a polyoxyethyleneglycerol ester, a polyoxyethylene fatty acid ester, a polyoxypropylene fatty acid ester, a polyoxyethylene/polyoxypropylene block copolymer, an alkylpolyglycoside, a benzalkonium chloride and/or a cetylpyridinium chloride or a combination of these surfactants.

17. The pharmaceutical preparation according to claim 15, wherein the surfactant is polyvinylpyrrolidone K25, polyoxyethylene-20-sorbitan monolaurate or polyoxyethyleneglycerol trioleate or a combination of these surfactants.

18. The pharmaceutical preparation according to claim 16 wherein the surfactant is present in a concentration of between 0.001 and 5% (m/m).

19. The pharmaceutical preparation according to claim 18, wherein the surfactant is present in a concentration of between 0.02 to 0.2% (m/m).

20. The pharmaceutical preparation according to claim 18, wherein the surfactant is present in a concentration of 0.3 to 2.5% (m/m).

21. The pharmaceutical preparation according to claim 8, wherein the cosolvent is present in a concentration of 8 to 12% (m/m) based on the formulation as a whole.

22. The pharmaceutical preparation according to claim 18 wherein the surfactant is present in a concentration of between 0.01 to 3% (m/m).

23. Pharmaceutical preparation according to claim 19, wherein the surfactant is present in a concentration of between 0.05 to 0.15% (m/m).

24. The pharmaceutical preparation according to claim 20, wherein the surfactant is present in a concentration of 0.4 to 2% (m/m).

25. The pharmaceutical preparation according to claim 24, wherein the surfactant is present in a concentration of 0.5 to 1.5% (m/m).

26. The pharmaceutical preparation according to claim 25, wherein the surfactant is present in a concentration of 0.75 to 1.25% (m/m).

27. The pharmaceutical preparation according to claim 2, wherein the active substance combination contains one or more active substances selected from among the anticholinergics, betamimetics, steroids, phosphodiesterase IV inhibitors, LTD4-antagonists, EGFR-kinase inhibitors, antiallergics, ergot alkaloid derivatives, triptanes, CGRP antagonists and phosphodiesterase-V inhibitors.

28. The pharmaceutical preparation according to claim 27, wherein the active substance combination contains beclomethasone, budesonide, cromoglycic acid, fenoterol, flunisolide, fluticasone, ipratropium, nedocromil orciprenaline, oxitropium bromide, reproterol, salbutamol, salmeterol (albuterol), terbutaline, N-[[2,2-dimethyl-4-(2-oxo-2H-pyridin-1-yl)-6-trifluoromethyl-2H-1-benzopyran-3-yl]methyl]-N-hydroxy-acetamide, the esters, salts and/or solvates thereof.

29. The pharmaceutical preparation according to one of claims 1, 11 or 16 wherein it contains the active substance combination salbutamol sulphate (albuterol sulphate) and ipratropium bromide monohydrate.

30. A metered-dose aerosol containing a pharmaceutical preparation according to claim 1.
